(19) United States
(12) Patent Application Publication
(10) Pub. No.: US 2004/0209267 A1
(43) Pub. Date: Oct. 21, 2004

(54) Method for identifying interaction between proteins and dna fragments of a genome

(76) Inventors: Stefan Beyer, Braunschweig (DE); Ursula Bilitewski, Braunschweig (DE); Joop Van Den Heuvel, Braunschweig (DE)

Correspondence Address: James P Zeller, Marshall Gerstein & Borun, 6300 Sears Tower, 233 South Wacker Drive, Chicago, IL, 60606-6357, US

(21) Appl. No.: 10/480,713

(22) PCT Filed: Jun. 11, 2002
(86) PCT No.: PCT/EP02/06406

(30) Foreign Application Priority Data
Jun. 12, 2001 (DE) 101 28 321.0

(51) Int. Cl. C12Q 1/68; C12P 19/34

(52) U.S. Cl. 435/6; 435/91.2

(57) ABSTRACT

The invention relates to a method of identifying interactions between proteins and DNA fragments of a genome, wherein at least one protein is incubated in vitro with at least one DNA fragment of a genome and the protein and/or DNA sequences that have interacted are then identified.

### SUBJECT OF THE INVENTION

The present invention relates to a new method for the parallel in vitro identification of DNA-protein interactions, that is to say for proteins of unknown function it is possible to find potential DNA-binding sites in the genome of organisms. For that purpose, the proteins being investigated are incubated with DNA fragments that represent the entire genome and then the sequences at which specific binding takes place are identified. The DNA fragments are preferably used in immobilised form and the binding of the protein being investigated is detected. It is thereby possible to identify upstream regulatory elements of genes and operons recognised by DNA-binding proteins, such as, for example, transcription factors. The clarification of regulation networks in complex biological systems is made simpler and quicker. For example, this invention can be utilised to describe the function and interrelationship of regulons and modulons in bacterial systems.

### PRIOR ART

a) Detection of DNA-Binding Proteins

The detection of DNA-protein interactions can be effected either in vivo, in vitro or by a combination of both experimental systems. The investigations usually need to be confined to one or only a small number of interacting and previously identified DNA-protein binding partners. By linking a so-called in vivo footprint to in vitro DNA microarray analyses it is possible, starting from a known or putative DNA-binding protein, also to test complex DNA mixtures for interactions. A pure in vitro technique for the global analysis of DNA-protein interactions within a sequenced or non-sequenced genome, as is the subject of this invention, has not been described hitherto.

In in vivo analyses, the effect of a DNA-binding protein in conjunction with a possible DNA-binding site on the transcription rate of an upstream gene in cis is determined. The mRNA formed is detected and optionally quantified, for example by radioactively labelling the mRNA during the in vivo transcription or subsequently hybridising the mRNA with radioactively labelled or fluorescent-labelled probes. When coupled systems are used in so-called reporter gene analyses, the transcription rate can be correlated indirectly with the amount of protein translated by that mRNA or with the enzyme activity derivable therefrom.

In in vitro detection methods for protein-DNA interactions, the DNA partner is generally radioactively labelled. Protein and DNA are incubated in solution under various conditions and then retained as a pair either by a nitrocellulose membrane (filter binding) or immunoprecipitated with an antibody (McCay assay). A further customary method is the gel retardation assay in which the electrophoretic flow behaviour of protein-bound DNA is compared with that of non-bound DNA. With the aid of different incubation conditions, such as salt concentration, pH value, competitor DNA etc., conclusions can be drawn as to the specificity and binding constants between protein and DNA fragment.

Chromatin immunoprecipitation with subsequent DNA microarray hybridisation (Iyer, V. R., Horak, C. E., Scafe C. S., Botstein D., Snyder, M., Brown, P. O., (2001) Genomic binding sites of the yeast cell-cycle transcription factors SBF and MBF, Nature 409, 533-538) is based on the formaldehyde- or glutaraldehyde-catalysed covalent linkage of DNA to cellular proteins in an intact cell system. DNA-binding proteins are bound preferentially to the DNA as a result of the spatial proximity. The DNA from the cells so treated is extracted, cut into fragments (for example by shearing) and then immunoprecipitated with the aid of an antibody directed against the DNA-binding protein being investigated. After separation of the DNA-binding protein, the DNA can be amplified, labelled and hybridised against DNA spotted onto microarrays. In comparison with a control, for example a deletion mutant relating to the protein being investigated, it is thus possible for specifically bound DNA fragments to be identified in parallel. The detection limit of this method is directly dependent upon the presence, or the amount present, of the regulatory protein being investigated, the intracellular concentration of which is generally very low.

b) Methods of Immobilising DNA or Proteins

For coupling DNA fragments (or oligonucleotides, PCR products), various methods are available that exploit different properties of nucleic acids:

nucleic acids are polymers and therefore bind purely absorptively to suitable support materials, such as, for example, membranes.

nucleic acids are always negatively charged by virtue of the phosphate radicals of the nucleotides. Accordingly, they can bind to positive support surfaces by means of purely electrostatic forces. This is exploited by the use of supports which are functionalised with amino groups. (for example polylysine-coated supports, or supports which have been modified with aminopropyl-triethoxysilane (APTS)), or membranes having positively charged side groups. Both methods (pure adsorption, or adsorption assisted by electrostatic forces) have the distinguishing features that they are simple to carry out, because the simple incubation of the support with the nucleic acid is sufficient for coupling, and they retain the functionality of the bound molecules to an extremely great extent, because they do not require further manipulation of the biomolecules. Such methods are therefore suitable for combination with spotters etc. and thus for the preparation of arrays.

The adhesion of nucleic acids to the support is improved when the bonds between support and biomolecule are covalent. Nucleic acids have a phosphate group at their 5′-end. That group can be activated (e.g. by carbodiimides and succinimides), so that chemical coupling to amino groups can take place. An alternative to that method, which has still been little tested hitherto, is the use of modified nucleic acids, especially nucleic acids having an amino group or a biotin radical at one end. Such nucleic acids (oligonucleotides) are commercially available and can be incorporated by means of polymerase reactions into the nucleic acid being investigated. Amino-functionalised nucleic acids quickly bind to aldehyde-modified supports (forming Schiff's bases) which, in the form of glass supports, are likewise commercially available. Alternatively, they can be coupled to epoxide-functionalised supports (modified, for example, by suitable silanisation reagents) or to supports having carboxy groups (after activation with carbodiimide and succinimide). The use of biotinylated nucleic acid allows stable coupling to avidin- or streptavidin-modified supports. In particular, couplings by way of reactions of amino groups with aldehyde groups or by way of biotin-(strept)avidin take place so quickly that they are also suitable for the preparation of arrays with spotters.

The coupling of nucleic acids (or proteins, e.g. avidin or streptavidin) frequently requires modification of the support surface with suitable functional groups (e.g. amino, aldehyde, epoxy, carboxy groups). In some cases such supports are commercially available, while in others those modifications must be carried out oneself, which is generally effected by reaction of the support with suitable silanisation reagents. In that case no special demands are made of the support, so that planar glass supports can be silanised equally as well as glass beads, or metal supports.

Further modification of support surfaces is frequently described in order to suppress non-specific interactions between subsequently added molecules and the support. Such further modifications include saturation of the support surfaces with inert proteins, such as bovine serum albumin, and also coating with polymers, such as dextrans, or polyacrylamide gel, which may also serve as a basis for the immobilisation and thus increase the loading capacity of the surface. The immobilisation principles described above can also be used for proteins, because proteins, by virtue of the side groups of the amino acids, have sufficiently functional groups especially for covalent couplings. Since the charge of proteins is not so uniform as that of nucleic acids, immobilisation as a result of electrostatic interactions is not so significant, but pure adsorption reactions, which also include hydrophobic interactions, often result in stable protein layers.

c) Detection of Affinity Reactions

The classic detection of bound proteins or nucleic acids is effected by way of inserted radioactive isotopes, such as, for example, ³²P For some applications, that method continues to be the most sensitive method and is therefore the method of choice (e.g. in investigations for the expression of genes from a small amount of mRNA). As an alternative, especially in the field of DNA analysis, fluorescence detection has become established. Here, in polymerase reactions there are used nucleotides that have been labelled with fluorescent dyes, so that the reaction products likewise carry a fluorescent marker. Their binding to a support, for example after a hybridisation reaction, can then be detected with the aid of fluorescence scanners. Proteins too can be labelled with fluorescent dyes without difficulty, as is known especially for the fluorescence labelling of antibodies. Care should be taken that the binding properties of the protein are not altered by the labelling. For example, the coupling of the fluorescent dye in the region of the binding site of the protein could prevent binding, or the coupling of strongly hydrophobic dyes can result in additional non-specific hydrophobic interactions with a support surface.

In addition to those detections by way of directly detectable markers (radioisotopes, fluorescent dyes), detection by way of secondary reactions of the markers is also known. For example, as markers there are also used enzymes, the reaction products of which are detected (enzyme immunoassay principle), or labels for which binding partners exist (e.g. biotin with streptavidin peroxidase and enzymatic reaction; digoxigenin with an enzyme-labelled antibody and enzymatic secondary reaction; His₆ with the corresponding antibody). Such markers are suitable both for the detection of bound nucleic acids and for the detection of bound proteins.

The most elegant detection methods, however, are based on the direct, marker-free detection of an affinity reaction. They allow not only detection of the binding of an unmodified binding partner, but also quantification of that binding and monitoring of the binding reaction in real time and thus the determination of kinetic constants, that is to say the association and dissociation constants. Those methods are essentially based on the fact that as a result of the binding reaction to a partner immobilised on a support, the mass load on that support or/and the thickness of the layers on that support changes. The change in layer thickness can be monitored by interferometric, that is to say optical methods (Rifs principle); for the detection of changes in the mass load both optical (surface plasmon resonance (SPR), interferometers, “resonant mirrors”, grating couplers) and piezoelectric methods are used. The sensors (transducers) on which one reaction partner of the affinity reaction is immobilised is accordingly frequently a glass support (interferometer, “resonant mirror”, grating coupler), which may be coated with gold or silver (surface plasmon resonance), or a gold electrode (piezoelectric measurements).

Whereas radioisotopes and fluorescent dyes can be detected after the support has dried, enzyme markers require incubation with the enzyme substrate solutions, in which case (with suitable substrates) insoluble precipitates can form at the site of the enzyme reaction. The marker-free detection of biochemical reactions has hitherto predominantly been carried out in the presence of a liquid phase, it being possible for surface plasmon resonance, interferometer and piezoelectric measurements to be carried out also in the gaseous phase.

For the evaluation of arrays, locally resolved detection is required. This has become well established for radioactive and fluorimetric measurements. The detection of insoluble products of enzyme reactions can also be effected with suitable densitometric scanners (analogous to the evaluation of Western Blots). Parallel evaluation in the case of marker-free methods has been limited hitherto. Commercial SPR apparatus have only up to four channels for the parallel investigation of up to four affinity reactions. Here too, however, systems for the parallel evaluation of more reactions are in development.

Hitherto, arrays have been used primarily for the detection of DNA-DNA interactions (expression analysis or sequencing with gene chips). The need to clarify the functions of proteins encoded by the genes has brought the development of protein arrays further into the limelight, especially the detection of proteins by specific antibodies or similar binding proteins (protein A/G; receptors), or the detection of enzyme activities. DNA-protein interactions with DNA fragments immobilised in array format have not been described hitherto. There are, however, publications relating to the detection of DNA-protein interactions, but either immobilised proteins are used (UPA; radioactive detection; many proteins are investigated simultaneously for binding of a DNA sequence; no modulation of the protein-binding activity by additives; see 2.b), or the binding of only a single DNA-protein pair is observed (piezoelectric detection; immobilised double-stranded 29-mer; see 1b).

### DESCRIPTION OF THE INVENTION

The rapidly increasing availability of fully sequenced genomes shows that often more than 50% of all annotated genes cannot be ascribed a function or can be ascribed only a putative function. Some of those genes or gene products are transcription factors which have been annotated as such on the basis of protein comparisons. The proportion of those only putative regulatory proteins increases as the complexity of the organisms increases.

The present invention enables a function to be ascribed to proteins, or genes, the biological function of which has been unknown or only partially known hitherto. The invention allows for the first time a comprehensive, parallel in vitro identification of DNA-protein interactions and is independent of the expression of the regulatory protein being investigated in the actual host. It is thus possible, unaffected by environmental conditions, to clarify global regulation networks in organisms in order to test those networks for their suitability for use in the modulation of metabolic pathways. Subsequent “genetic engineering” allows optimisation of, for example, fermentation processes for obtaining enzymes, chemicals and pharmaceutically useful substances. In addition, the regulatory networks revealed on the basis of this invention can lead to the identification of new “active ingredient targets” for the treatment of diseases in human beings, animals and plants.

The problem underlying the invention is therefore solved by a method of identifying interactions between proteins and DNA fragments of a genome, in which at least one protein is incubated in vitro with at least one DNA fragment of a genome and the protein and/or DNA sequences that have interacted are then identified.

In the method according to the invention it is possible to carry out incubation with DNA fragments that represent the entire genome of an organism.

In the method according to the invention, the DNA fragments can represent the genome of a prokaryote or eukaryote, especially a bacterium, a yeast or a fungus.

In the method according to the invention, DNA fragments in the form of a “shot-gun” DNA library can be used as starting material.

Furthermore, in the method according to the invention, DNA fragments in the form of a DNA vector library, especially in the form of a DNA plasmid library, can be used as starting material.

In the method according to the invention, the DNA vector library can be provided using Escherichia coli clones.

In the method according to the invention, the DNA fragments in the form of a DNA library or in the form of a DNA vector library can be arranged in microtitre plates.

In that case the DNA fragments can be in a multiply duplicated arrangement.

Furthermore, in the method according to the invention the vector DNA of the library can be double-stranded and immobilised on a solid matrix.

In that case the vector DNA can be provided in an ordered or systematic way.

As solid matrix, in the method according to the invention an optionally functionalised support can be provided which is a glass support, a membrane or a gel.

In that case, for the method according to the invention there can be provided a functionalisation of the support that is able to couple the immobilised DNA fragments, especially to bind them covalently or electrostatically.

In the method according to the invention, the optionally functionalised support can be provided on a planar supporting element, in a column or in or on a capillary.

Furthermore, in the method according to the invention there can be used a protein which is derived from the represented genome.

In that case, for the method according to the invention the derived protein can have been expressed heterologously in a prokaryotic, especially a bacterial, expression system or in a eukaryotic expression system.

For example, according to the invention the derived protein can have been expressed with an artificial epitope tag, especially a histidine hexapeptide as an epitope tag.

In the method according to the invention, the incubation can be carried out simultaneously on all immobilised DNA fragments batchwise or in throughflow.

Furthermore, in the method according to the invention, the incubation can be carried out sequentially, especially in the case of immobilisation in a column or in or on a capillary.

Furthermore, in the method according to the invention, the interacting partners, DNA fragment and protein, can be covalently linked to one another, especially with an aldehyde, preferably formaldehyde or glutaraldehyde.

In the method according to the invention, the interacting protein can be detected by:

detecting the protein with the aid of at least one antibody which is directed against the protein or the epitope tag of the protein,

detecting the protein by surface plasmon resonance, especially when the support is gold-coated,

detecting the protein with the aid of the resonant mirror method,

detecting the protein with the aid of an interferometer,

detecting the protein with the aid of piezocrystals or

detecting the protein with the aid of a marker, especially a fluorophore or a radioactive marker.

According to the invention, the DNA fragments can have a size of from 1.5 to 2.5 kb, from 1.0 to 2.0 kb, from 0.5 to 1.5 kb, from 0.3 to 0.8 kb or from 0.03 to 0.5 kb.

The method according to the invention can be carried out for identifying DNA-protein interactions in complex DNA fragment mixtures, especially for identifying DNA-binding sites for proteins of known or unknown function.

Furthermore, the method according to the invention can be carried out for identifying cis-arranged regulatory elements for the transcription of eukaryotic or prokaryotic genes.

Furthermore, the method according to the invention can be carried out for ascribing a function to genes and/or proteins that relate to gene regulation, especially for identifying transcription factors or genes that are regulated by transcription factors.

Furthermore, the method according to the invention can be carried out for clarifying regulation networks in complex biological systems, especially for optimising the culturing of prokaryotes or eukaryotes on the basis of regulation networks.

For example, the method according to the invention can be carried out for optimising fermentation processes with the aid of prokaryotes or eukaryotes on the basis of regulation networks.

Furthermore, the method according to the invention can be carried out for identifying targets for the development of active ingredients or methods of treating diseases.

A further embodiment of the invention relates to a microtitre plate having a DNA library or DNA vector library arranged therein for carrying out the method according to the invention.

A further embodiment of the invention relates to a solid matrix according to the invention having DNA fragments immobilised on the matrix.

A further embodiment of the invention relates to a planar supporting element according to the invention having a solid matrix according to the invention applied thereto.

The invention will be illustrated in greater detail below by way of examples.

### EXAMPLE 1

1) The setting-up of a “shot-gun” DNA plasmid library having an average insert size of either 2 kb, 1.5 kb, 1 kb or 0.5 kb of the bacterium being investigated. The DNA library covers the genome of the bacterium being investigated several times. The E. coli clones containing the plasmid library are arranged in microtitre plates, multiply duplicated and kept.

2) The plasmid DNA of the library is immobilised in an ordered manner in the form of a double strand on a solid matrix. The matrix used can be a treated/coated glass surface, a membrane or a gel of different materials. The treatment or coating of the glass surface can be carried out in order to allow the coupling of the DNA (covalent or electrostatic), to suppress non-specific binding (see 5.) or to facilitate detection (coating with e.g. a gold layer for detection by means of surface plasmon resonance). Supports other than glass supports may be used when detection methods other than optical methods are chosen. Alternatively, it is also possible for only the insert DNA of the recombinant plasmids to be immobilised in a directed manner. The amplification of the insert DNA can be effected, for example, using PCR. The matrix can be located on a planar support, or in a column or on the wall of a capillary.

3) The choice of protein to be investigated is made by computer-assisted analysis of existing DNA and protein sequences, for example from fully or partially sequenced genomes. A comparison of derived amino acid sequences of ORFs with protein data banks can provide the first pointers to DNA-binding proteins. The allocation of the derived proteins to so-called COGs (clusters of orthologous groups) or the presence of specific structural motifs (for example “helix-turn-helix” motifs) can likewise provide predictions of a potential DNA-binding activity.

4) The gene/gene product selected under 3) is expressed heterologously in a bacterial or eukaryotic expression system with or without an artificial epitope tag (e.g. histidine hexapeptide) and then purified in the native state.

5) The protein in buffer solution is incubated with the double-stranded DNA immobilised on a matrix. Non-specific interactions of the protein with the matrix or the DNA are suppressed by suitable surface treatments or washing steps or rendered visible by means of competition experiments. The incubation can be carried out simultaneously on all immobilised DNA samples batchwise or in throughflow, or sequentially (preferred in the case of immobilisation of the DNA in columns or capillaries and corresponding throughflow systems).

6) In the system described under 5), after incubation of the protein with the immobilised DNA samples and after the appropriate washing operations the interacting partners are covalently linked to one another optionally with the aid of crosslinkers (e.g. formaldehyde or gluraldehyde). The detection of the regulatory protein is then effected by means of immunological methods using (enzyme, fluorescence or radioactively) labelled antibodies which are directed against the regulatory protein or the epitope tag present on the regulatory protein. When detection methods allowing marker-free measurement are used (e.g. surface plasmon resonance; “resonant mirrors”; interferometers; piezocrystals), the addition of the protein can be monitored directly without the need for antibodies. This is also the case when it is possible to label the protein being investigated in such a way (e.g. with a fluorophore or radioactively) that binding to the DNA is not affected. The important point is that detection is effected in such a manner that the signal can be allocated to a specific DNA fragment.

7) By means of the method described above, protein-DNA binding partners are identified with the aid of a heterologously expressed protein and a DNA library. The ordered plasmid library allows unambiguous allocation to genomic DNA regions of the organism being investigated. The sequencing of the inserts of the filtered-out plasmids enables the genes located in the immediate vicinity of the protein-binding region to be identified. In the case of fully sequenced genomes it is thus possible to identify DNA sequences having an influence on the regulation of genes and operons. The biochemical and biological characterisation of the DNA-protein interactions can then be effected using various “state-of-the-art” methods (e.g. by sequence comparison, marker gene analysis in combination with deletion studies, DNAse protection analysis, reporter gene analyses etc.).

### EXAMPLES 2 TO 3

Investigations relating to the regulation of morphological and physiological differentiation in the case of bacteria that produce secondary substances, such as, for example, myxobacteria.

Identification of metabolic-pathway-specific and pleiotropic regulators and the genes and operons influenced transcriptionally thereby, i.e. regulons and modulons.

### EXAMPLES 4 TO 8

Examples analogous to the prior art for the regulation of modulons by certain DNA-binding proteins in E coli are inter alia:

LexA modulates the expression of many regulons of the SOS system.

TyrA controls eight operons bifunctionally as repressor and activator.

FnrR is involved in the regulation of more than thirty transcription units.

LrpR influences the transcription of eight operons.

PhoPQ is involved in the regulation of at least seven operons.

### SELECTED LITERATURE

1. DNA chips:

a) Nature Genet. 21 (1999) Suppl.

b) K. Niikura, K. Nagata, Y. Okahata, Quantitative detection of protein binding onto DNA by using a quartz-crystal microbalance, Chem. Lett. 1996, 8634

c) D. Guschin, G. Yershov, A. Zaslavsky, A. Gemmell, V. Schick, D. Proudnikov, P. Arenkov, A. Mirzabekov, Manual Manufacturing of oligonucleotide, DNA, and protein microchips, Anal. Biochem. 250, 1997, 203-11

2. Protein arrays

a) P. Arenkov, A. Kukhtin, A. Gemmel, S. Voloshchuk, V. Cupeeva, A. Mirzabekov, Protein Microchips: Use for immunoassay and enzymatic reactions, Anal. Biochem. 278, 2000, 123-31

b) H. Ge, UPA, a universal protein array system for quantitative detection of protein-protein, protein-DNA, protein-RNA and protein-ligand interactions, Nucleic acids research 28, 2000, e3

c) A. Lueking, M. Horn, H. Eickhoff, K. Bussow, H. Lehrach, G. Walter, Protein microarrays for gene expression and antibody screening, Anal. Biochem. 270, 1999, 103-111

d) G. MacBeath, S. L. Schreiber, Printing proteins as microarrays for high-throughput function determination, Science, 289, 2000, 1760-1763

1. Method of identifying interactions between proteins and DNA fragments of a genome, comprising the steps of incubating at least one protein in vitro with at least one DNA fragment of a genome, and identifying the protein and/or DNA sequences that have interacted.

2. Method according to claim 1, comprising carrying out incubation with DNA fragments that represent the entire genome of an organism.

3. Method according to claim 1, wherein the DNA fragments represent the genome of a prokaryote or eukaryote.

4. Method according to claim 1, comprising using a “shot-gun” DNA library as starting material.

5. Method according to claim 1, comprising using a DNA vector library, as starting material.

6. Method according to claim 1, comprising providing the DNA vector library using Escherichia coli clones.

7. Method according to claim 1, comprising providing the DNA fragments in the form of a DNA library or in the form of a DNA vector library arranged in microtitre plates.

8. Method according to claim 7, wherein the DNA fragments are in a multiply duplicated arrangement.

9. Method according to claim 4, wherein the vector DNA of the library is double-stranded and immobilized on a solid matrix.

10. Method according to claim 9, comprising providing the vector DNA in an ordered or systematic way.

11. Method according to claim 9, wherein the solid matrix comprises a support which is a glass support, a membrane, or a gel.

12. Method according to claim 11, wherein the support is functionalized to couple the immobilized DNA fragments.

13. Method according to claim 11, comprising providing the support on a planar supporting element, in a column, or in or on a capillary.

14. Method according to claim 1, comprising using a protein derived from the represented genome.

15. Method according to claim 14, wherein the derived protein has been expressed heterologously in a prokaryotic, expression system or in a eukaryotic expression system.

16. Method according to claim 15, wherein the derived protein has been expressed with an artificial epitope tag.

17. Method according to claim 1, wherein the DNA fragments are immobilized, comprising carrying out the incubation simultaneously on all immobilized DNA fragments batchwise or in throughflow.

18. Method according to claim 1, comprising carrying out the incubation sequentially.

19. Method according to claim 1, wherein the interacting DNA fragment and protein are covalently linked to one another.

20. Method according to claim 1, comprising detecting the interacting protein by a method selected from the group consisting of:
detecting the protein with the aid of at least one antibody which is directed against the protein or an epitope tag of the protein,
detecting the protein by surface plasmon resonance,
detecting the protein with the aid of the resonant mirror method,
detecting the protein with the aid of an interferometer,
detecting the protein with the aid of piezocrystals, or
detecting the protein with the aid of a marker.

21. Method according to claim 1, wherein the DNA fragments have a size of from 1.5 kb to 2.5 kb.

22. Method according to claim 1, comprising carrying out the method for identifying DNA-protein interactions in complex DNA fragment mixtures.

23. Method according to claim 1, comprising carrying out the method for identifying cis-arranged regulatory elements for the transcription of eukaryotic or prokaryotic genes.

24. Method according to claim 1, comprising carrying out the method for ascribing a function to at least one of genes and proteins that relate to gene regulation.

25. Method according to claim 1, comprising carrying out the method for clarifying regulation networks in complex biological systems.

26. Method according to claim 25, comprising carrying out the method for optimizing fermentation processes with the aid of prokaryotes or eukaryotes on the basis of regulation networks.

27. Method according to claim 1, comprising carrying out the method for identifying targets for the development of active ingredients or methods of treating diseases.

28. Microtitre plate having a DNA library or DNA vector library arranged therein for carrying out a method according to claim 1.

29. Solid matrix having DNA fragments immobilized on the matrix.

30. Planar supporting element having a solid matrix according to claim 29 applied thereto.

31. Method according to claim 3, wherein the DNA fragments represent the genome of a prokaryote or eukaryote selected from the group consisting of bacteria, yeast, and fungi.

32. Method according to claim 5, comprising using a DNA plasmid library as starting material.

33. Method according to claim 12, wherein the functionalized support binds the immobilized DNA fragments covalently.

34. Method according to claim 12, wherein the functionalized support binds the immobilized DNA fragments electrostatically.

35. Method according to claim 15, wherein the derived protein has been expressed in a bacterial expression system.

36. Method according to claim 16, wherein the derived protein has been expressed with a histidine hexapeptide as an epitope tag.

37. Method according to claim 18, wherein the DNA fragments are immobilized in a column or in or on a capillary.

38. Method according to claim 19, wherein the interacting DNA fragment and protein are covalently linked to one another with an aldehyde.

39. Method according to claim 38, wherein the aldehyde is selected from the group consisting of formaldehyde and glutaraldehyde.

40. Method according to claim 20, wherein the DNA is immobilized in a gold-coated support, and the interacting protein is detected by surface plasmon resonance.

41. Method according to claim 20, comprising detecting the protein with the aid of a marker selected from the group consisting of fluorophore markers and radioactive markers.

42. Method according to claim 1, wherein the DNA fragments have a size from 1.0 kb to 2.0 kb.

43. Method according to claim 1, wherein the DNA fragments have a size from 0.5 kb to 1.5 kb.

44. Method according to claim 1, wherein the DNA fragments have a size from 0.3 kb to 0.8 kb.

45. Method according to claim 1, wherein the DNA fragments have a size from 0.03 kb to 0.5 kb.

46. Method according to claim 22, comprising carrying out the method for identifying DNA-binding sites for proteins of known function or unknown function.

47. Method according to claim 24, comprising carrying out methods for identifying transcription factors or genes that are regulated by transcription factors.

48. Method according to claim 25, comprising carrying out the method for optimizing the culturing of prokaryotes or eukaryotes on the basis of regulation networks.

49. Solid matrix according to claim 29, wherein the DNA is double-stranded and represents a “shot-gun” DNA library.

50. Planar supporting element according to claim 30, wherein vector-DNA of a “shot-gun” DNA library is double-stranded and immobilized on the solid matrix.
